(19) United States
(12) Plant Patent Application Publication
(10) Pub. No.: US 2019/0313562 P1
(43) Pub. Date: Oct. 10, 2019

(54) Apple tree variety denominated SPINK754

(50) ;

(71) Applicant: IVU California LLC, Brentwood, CA (US)

(72) Inventor: Alejandro Navarro, Curico (CL)

(21) Appl. No.: 15/932,712

(22) Filed: Apr. 10, 2018

(51) Int. Cl. A01H 6/74

(52) U.S. Cl. PLT/161

(57) ABSTRACT

A new and distinct variety of apple tree is described and denominated SPINK754. The new variety exhibits an attractive and intense color, harvesting 1 to 2 weeks prior to the standard variety, and strong post-harvest preservation when grown under the conditions prevailing in the central valley of Chile.

### RELATED APPLICATIONS

Not Applicable.

### STATEMENT REGARDING FEDERALLY SPONSORED RESEARCH OR DEVELOPMENT

Not Applicable.

Latin name: Malus domestica Borkh.

Variety denomination: SPINK754.

### ORIGIN AND ASEXUAL REPRODUCTION

The present disclosure relates to a new and distinct variety of apple tree, Malus domestica, that has been denominated SPINK754, and more specifically to an apple tree variety that is principally characterized by an attractive and intense color, harvesting one to two weeks prior to the Cripps Pink variety, and presents better post-harvest preservation at its orchards of origin in the central valley of Chile. Attractive color and good handling and storage characteristics are well known to be important commercial factors when it comes to apples or other fruits. The new variety is further characterized by greater, more uniform starch content than Cripps Pink, and better resistance to internal browning post-harvest.

In March, 2008, a mutation of a branch of a Cripps Pink tree was selected on rootstock MM106 in a 20 hectare orchard located in the Curicó valley, central zone of Chile, located at 35 latitude south. The normal harvest date of Cripps Pink at this geographic location is one to two weeks later than the selected mutation. The mutation presented an intense pink coloration covering approximately 80% of the fruit, a greater degree of coloration than the approximately 60% coverage found on other fruit in the orchard.

In 2009 a total of ten trees of the new variety were propagated asexually by grafting onto M9 T337. The first fruits were obtained in 2011. The new variety was evaluated from 2011 to 2016 and determined to be stable. It was also determined that the new variety presents a better post-harvest life in the central valley of Chile, where the Cripps Pink variety exhibits high internal browning damage to the fruit after harvest. Fruits of the new variety either lacked this problem entirely or presented the problem to a very small degree.

### SUMMARY OF THE INVENTION

The SPINK 754 apple tree is characterized by an attractive and intense color, an earlier harvesting time than the Cripps Pink variety, and a strong diminishment in internal browning of fruit stored for more than five months. The fruit is ripe for harvesting and shipment during approximately mid-April under conditions prevailing in the central valley of Chile.

### BRIEF DESCRIPTION OF THE ILLUSTRATIONS

The accompanying photographs consist of color photographs as follows:

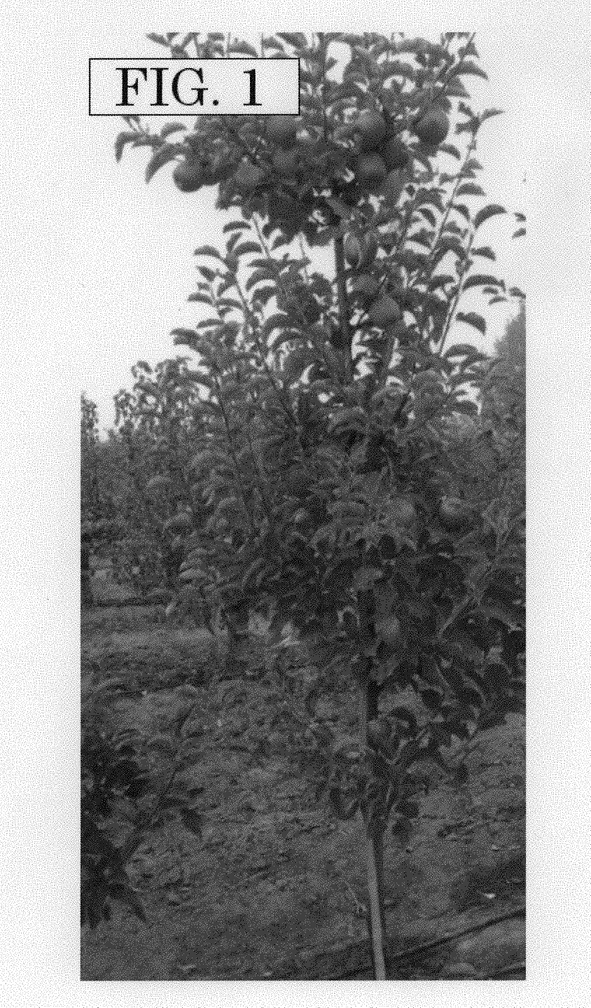

FIG. 1 depicts the new variety growing on or about Apr. 5, 2014 in the central valley of Chile.

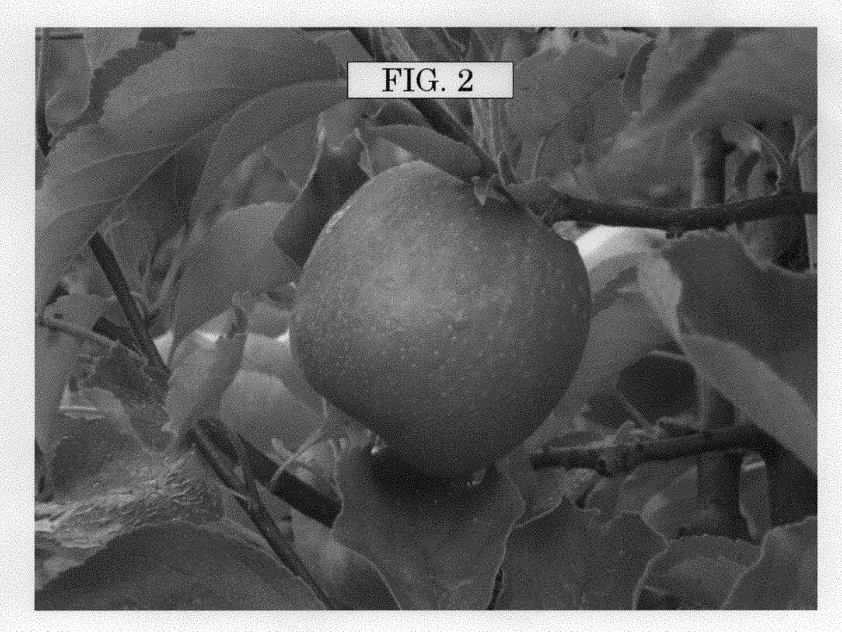

FIG. 2 is a close view of the fruit of the new variety, growing on or about Apr. 5, 2014 in the central valley of Chile.

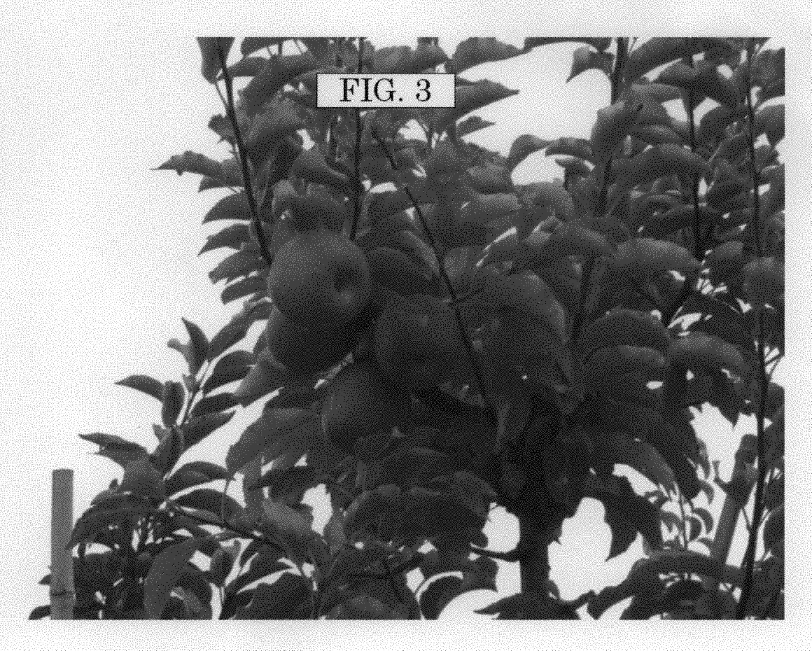

FIG. 3 is a cluster of fruit of the new variety, growing on or about Apr. 5, 2014, in the central valley of Chile.

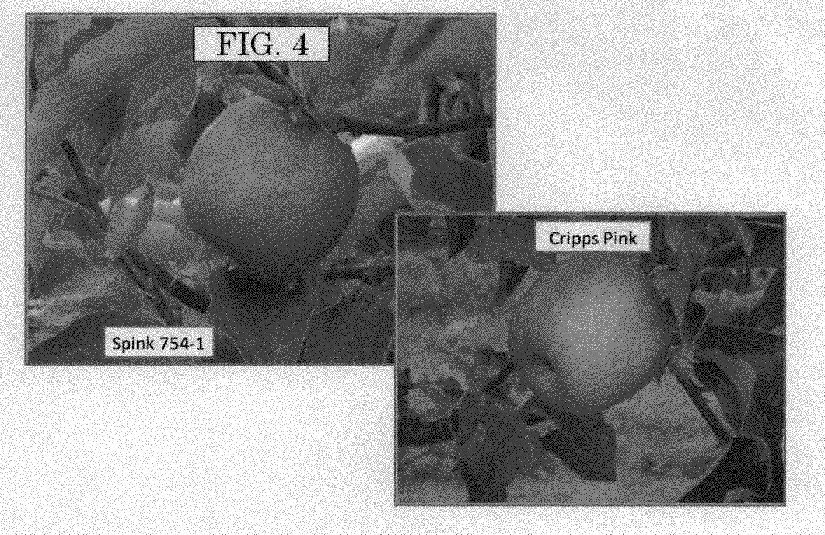

FIG. 4 is a comparison of the fruit of the new variety, and that of Cripps Pink, both growing on or about Apr. 5, 2014 in the central valley of Chile.

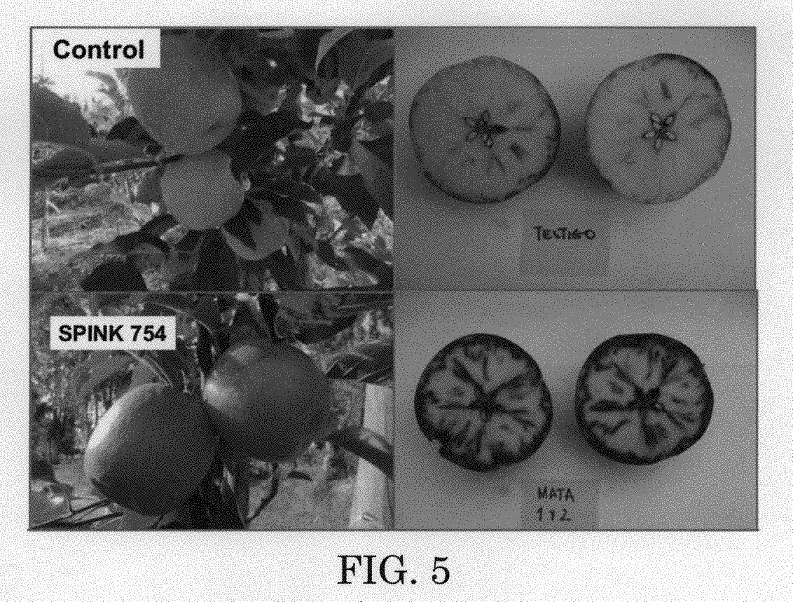

FIG. 5 is a comparison of the starch content of a Cripps Pink control and the fruit of the new variety, both grown in 2011 in the central valley of Chile.

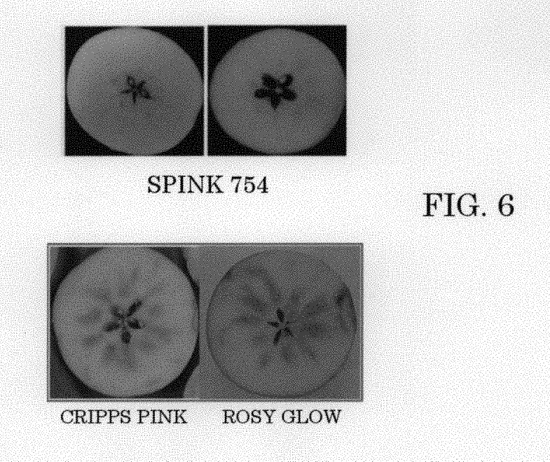

FIG. 6 is a comparison of internal browning of the new variety and that of the Cripps Pink and Rosy Glow varieties, with each fruit shown after about 150 days of cold storage.

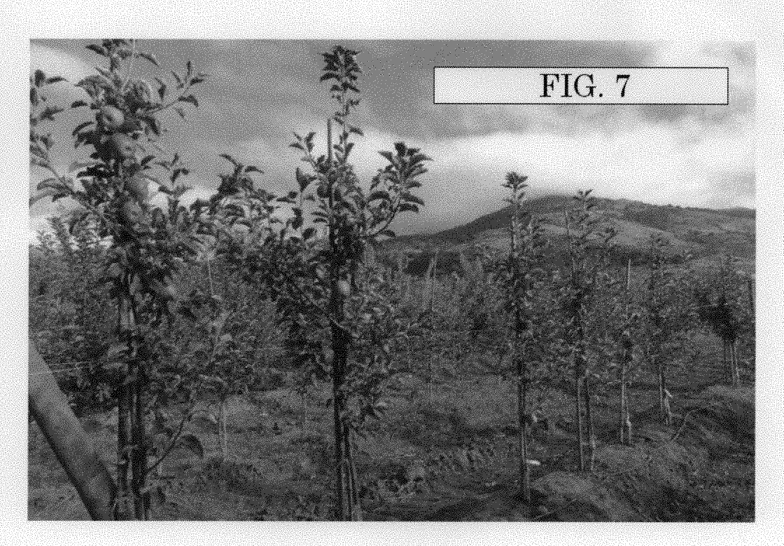

FIG. 7 is an early tree of the new variety showing its first fruit on or about Apr. 15, 2011.

### DETAILED DESCRIPTION OF THE INVENTION

Referring to the botanical description of the new and distinct variety, the following traits and characteristics have been observed in representative plants of the new variety grown in the central valley of Chile.

### BOTANICAL DESCRIPTION

Tree:
   Type.—Ramified; extended habit.
   Vigor.—Medium.
   Fruiting.—Only in long stalks.
Branches:
   Thickness.—Medium thickness after one year, observed at center of middle internode.
   Internode length.—Medium length after one year, observed in middle third of branch.
   Color.—Reddish-brown at portion exposed to sunlight, after one year.
   Pubescence.—Medium, after one year.
   Number of lenticels.—Medium.
Leaves:
   Support from branch.—Horizontal.
   Length.—Medium.
   Width.—Medium.
   Relationship between length and width.—Large.
   Intensity of green color.—Dark.
   Incisions on edge.—Bicrenate.
   Pubescence on bottom surface.—Absent or weak.
   Petiole length.—Long.
   Petiole, extension of coloring of anthocyanins.—Medium.
Flowers:
   Color.—Predominantly light pink in cocoon phase.
   Diameter.—Large, when petals extended.
   Petal disposition.—Intermediate.
   Position of stigma in relation to anthers.—At the same level.
   Good pollinator.—Granny Smith.
Fruit:
   Extension of coloring of anthocyanin in young fruit.—Medium.
   Size.—Large to very large.
   Height.—High.
   Diameter.—Large.
   Relationship of height to diameter.—Large.
   Shape.—Cylindrical, generally.
   Ribbing.—Moderate.
   Crown at the end of the cup.—Moderate.
   Size of eye.—Large.
   Length of sepal.—Short.
   Bloom of epidermis.—Strong.
   Greasiness of epidermis.—Strong.
   Color of bottom.—Yellowish-green.
   Zone relative to color top.—Very large.
   Tone of color at top once bloom is removed.—Pinkish-red.
   Intensity of top color.—Dark.
   Distribution of top color.—Uniform.
   Zone of russeting around peduncular base.—Absent or small.
   Zone of russeting on face.—Absent or small.
   Zone of russeting of eye cavity.—Absent or small.
   Number of lenticels.—Medium.
   Size of lenticels.—Medium.
   Length of pedicle.—Short.
   Thickness of pedicle.—Medium.
   Depth of pedicle cavity.—Deep.
   Width of pedicle cavity.—Wide.
   Depth of eye cavity.—Deep.
   Width of eye cavity.—Wide.
   Firmness of pulp.—Very firm.
   Color of pulp.—Cream.
   Opening of loculus.—Closed or slightly open.
   Start time of flowering.—Very late.
   Time of harvest.—Late.
   Time to maturity for consumption.—Very late.

The new variety exhibits the characteristics shown and described when grown under the ecological conditions prevailing in the central valley of Chile. It is understood that variations of the usual magnitude and characteristics may be expected as a result of changes in growing conditions, location, fertilization, pruning, pest control, and horticultural management practices.

1. A new variety of apple tree denominated SPINK 754, and parts thereof, substantially as illustrated and described.
